SEQUENCE LISTING (2 sequences)

SEQ ID NO: 1 - DNA, 945 bp, Ochrobactrum anthropi
  CDS (1)..(945)
atgtgcaataattgccattacaccattcacggccggcatcatcatttcggctgggacaactcgttccagccggctgaaacggtcgcgcccggctcgaccctgaaattcgaatgtctggacagcggcgcaggccactatcatcgcggcagcacagtcgccgatgtgtcgacgatggatttttccaaggtcaatccggttaccggccccatcttcgtcgatggagccaaaccgggcgatgtcctgaaaatcaccatccaccagttcgagccatcaggcttcggctggacggcaaatattccgggcttcggtcttctcgccgacgacttcaaggaaccggcgctagcattgtggaactacaatcccacaacgctggagccagcactcttcggagagcgtgcgcgcgtgccgctgaagccgttcgccggaaccatcggcgtcgcaccggcggaaaagggcctgcattcggtcgtaccaccgcgtcgtgtcggcggcaatctcgacatccgcgatcttgcagccggaaccacgctttatctgccgatcgaagtcgaaggcgctttgttctccattggtgatacccatgcggcacagggcgacggcgaagtgtgcggcaccgccatcgaaagcgcgatgaatgtcgctctgacgctggatctcatcaaggatacgccactgaagatgccccggttcaccacgccggggccagtgacgcggcacctcgataccaagggttacgaagtcaccaccggtatcgggtccgatctgtgggaaggcgcgaaagccgccctctccaacatgatcgaccttctttgccagacgcagaacctcaacccggtggatgcctatatgctctgctcggcctgcggtgatctgcgtatcagcgaaatcgtcgatcagccgaactgggtcgtatcgttctacttcccgcgttccgttttcgaataa

SEQ ID NO: 2 - protein, 314 aa, Ochrobactrum anthropi
MCNNCHYTIHGRHHHFGWDNSFQPAETVAPGSTLKFECLDSGAGHYHRGSTVADVSTMDFSKVNPVTGPIFVDGAKPGDVLKITIHQFEPSGFGWTANIPGFGLLADDFKEPALALWNYNPTTLEPALFGERARVPLKPFAGTIGVAPAEKGLHSVVPPRRVGGNLDIRDLAAGTTLYLPIEVEGALFSIGDTHAAQGDGEVCGTAIESAMNVALTLDLIKDTPLKMPRFTTPGPVTRHLDTKGYEVTTGIGSDLWEGAKAALSNMIDLLCQTQNLNPVDAYMLCSACGDLRISEIVDQPNWVVSFYFPRSVFE

(19) United States
(12) Patent Application Publication
(10) Pub. No.: US 2005/0130140 A1
(43) Pub. Date: Jun. 16, 2005

(54) Process for preparing variant polynucleotides

(76) Inventors: Roelof Ary lans Bovenberg, Da Rotterdam (NL); Richard Kerkman, Nl Zandvoort (NL)

Correspondence Address: MORRISON & FOERSTER LLP, 3811 VALLEY CENTRE DRIVE, SUITE 500, SAN DIEGO, CA, 92130-2332, US

(21) Appl. No.: 10/484,680

(22) PCT Filed: Jul. 23, 2002
(86) PCT No.: PCT/EP02/08222

(30) Foreign Application Priority Data
Jul. 23, 2001 (EP) 01202822.1
Sep. 11, 2001 (EP) 01203458.3

(51) Int. Cl. C12Q 1/68; C12P 19/34

(52) U.S. Cl. 435/6; 435/91.2

(57) ABSTRACT

The present invention discloses a process for the preparation of variant polynucleotides using a reassembly process of preferably blunt-ended restriction enzyme fragments prepared form a starting population of heterologous polynucleotides in the presence of a thermostable ligase.

### BACKGROUND OF THE INVENTION

Protein engineering technology includes the creation of novel proteins by targeted modification(s) of known proteins. However, an approach directed to targeted modification is only applicable to proteins or protein families of which the three-dimensional structure of the protein or at least one member protein of the family has been resolved. Furthermore, many attempts to alter the properties of enzymes by this approach have failed because unexpected changes in the structure were introduced. If random mutagenesis is applied to create modified proteins, it appeared that successfully modified proteins often possessed amino acid substitutions in regions that protein modeling could not predict.

Various approaches have been developed to mimic and accelerate nature's recombination strategy to direct the evolution of proteins to more beneficial molecules. Direct evolution is a general term used for methods for random in vitro or in vivo homologous recombination of pools of homologous polynucleotides. Several formats are described, for instance random fragmentation followed by polymerase-assisted reassembly (WO 9522625), in vivo recombination (WO97107205, WO98/31837) or staggered extension of a population of polynucleotide templates (WO97/07205, WO98/01581). In this way an accumulation of beneficial mutations in one molecule may be accomplished.

The method of the present invention advantageously enables the random combination of mutated positions in a rapid, reproducible and highly controllable way. A further advantage of the method of the invention is that the recombination frequency is high and the chance to re-isolate the starting polynucleotide is low.

### DETAILED DESCRIPTION

The present invention provides a method for the preparation of a variant polynucleotide.

The method according to the invention comprises the steps of:
subjecting separate fractions of a population of polynucleotides to digestions with a restriction enzyme, preferably capable of generating blunt-ended fragments, combining the digests,
applying one or more cycles of denaturation, annealing and reassembly in the presence of a ligase,
optionally amplifying the reassembled polynucleotides,
preparing a library of the resulting variant polynucleotides,
screening said library of variant polynucleotides for a variant polynucleotide with a desired property.

A variant polynucleotide is defined herein as a polynucleotide differing in at least one position from any one of the members of the population of polynucleotides that forms the starting material for the process according to the invention.

The population of polynucleotides that forms the starting material for the process according to the invention comprises polynucleotide members that display a substantial homology to each other. A substantial homology is defined herein as a homology from 70-100%, preferably from 75-100%, preferably from 80-100%, preferably from 85-100%, more preferably from 90-100%, most preferably from 95-100%. A population of polynucleotides comprising polynucleotide members displaying a substantial homology for instance may be a population of polynucleotides wherein the polynucleotide members are identical polynucleotides, and/or are mutants of a parental polynucleotide and/or are members of a gene family.

A population of mutants derived from a parental polynucleotide may comprise different mutants, each individual mutant in the population differing in at least one position from the parental polynucleotide. A population of different mutants derived from a parental polynucleotide may be obtained by methods known in the art. For instance, the mutants may be obtained by classical random or site-directed mutagenesis techniques. A suitable random mutagenesis technique for instance is the error-prone PCR technique.

The population of mutants may comprise mutants that have been previously screened and selected for a certain desired property.

A population of members of a gene family typically contains different members of a gene family, i.e. polynucleotides displaying a considerable sequence homology, i.e. at least 70%, and having a similar function in an organism. For instance, such polynucleotides may encode related proteins originating from different strains, different species, different genera, different families. An example is the phytase gene family from the genus Aspergillus, displaying a homology of at least 90% within the species Aspergillus niger.

The starting population of polynucleotides may conveniently be subjected to the process of the invention when being cloned in a vector and/or as isolated fragments. In a situation that the starting population of polynucleotides is obtained by a prior screening and selection process, the vector may conveniently be an expression vector.

According to the method of the invention, separate fractions of the starting population of polynucleotides are subjected to digestion with a restriction enzyme.

The restriction enzyme used may be a single enzyme or may be a mixture of two or more enzymes. Preferably, the restriction enzyme(s) and/or the number of separate digestions is (are) chosen in such a way that the mutated positions and/or the regions of heterology as present within the members of the starting population of polynucleotides are located as much as possible on separate fragments. The separate restriction enzyme digests further are performed in such a way that the fragments obtained in the digests can serve as each other's template in a reassembly reaction upon combining the separately digested fractions.

In a preferred embodiment, each separate fraction of the starting population of polynucleotides is digested with a different restriction enzyme.

In another preferred embodiment, the restriction enzyme is capable of generating blunt-ended fragments. By using such a restriction enzyme, the chance of obtaining a substantial amount of the starting polynucleotide(s) after performing the process according to the invention is small.

After inactivating the restriction enzyme(s), the separate digests are combined and the combined digests are subjected to one or more cycles of denaturation, annealing and reassembly in the presence of a ligase.

The number of cycles may be chosen such that a detectable amount of recombined fragment is obtained. Preferably, 2-100 cycles are performed, more preferably 10-50 cycles, most preferably 20-40 cycles.

The ligase used preferably is a ligase capable of ligating single-strand nicks in a double stranded polynucleotide. Specifically, the ligase is capable of catalysing NAD-dependent ligation of adjacent 3′-hydroxylated and 5′-phosphorylated termini in duplex DNA structures. More preferably, the ligase used is a ligase substantially not capable of ligating blunt-ended polynucleotide fragments, i.e. a ligase with no or a low activity on blunt-ended polynucleotide fragments. Most preferably, the ligase used is a thermostable ligase. An especially preferred ligase is Ampligase (Epicentre). The products of the ligase-induced reassembly reaction may optionally be amplified by PCR.

A PCR as performed in the method of the invention may be performed following conditions generally known to the person skilled in the art. The conditions typically may depend on the primers and the enzyme used. It may further be an option to perform the PCR under error-prone conditions, i.e. under conditions that reduce the fidelity of nucleotide incorporation, thus randomly introducing additional mutations in the variant polynucleotides obtained by the method of the invention. Error-prone conditions may for instance be provided by independently varying the concentrations of manganese and dGTP in the PCR reaction. Typically, the mutagenesis rate may be raised by increasing the amount of manganese and/or dGTP in the PCR reaction.

The polynucleotide products of the reassembly reaction are cloned in a suitable vector, to enable the preparation of a library of variant polynucleotides. The choice of the vector will depend on the host wherein the library is propagated. Subsequently, the library of variant polynucleotides is screened with a suitable screening method to enable the selection of a variant polynucleotide with a desired property.

The method used for screening the library of variant polynucleotides is not critical for the invention. Typically, the method used will depend on a property of the polynucleotide of interest. If the polynucleotide of interest comprises a gene encoding a polypeptide, a suitable screening method may be directed to directly assay said polypeptide. A suitable screening method may further be directed to assay a primary or secondary metabolite if the polypeptide is an enzyme involved in the production of said primary or secondary metabolite, for instance an enzyme that is part of the biosynthetic pathway of said metabolite. Examples of such metabolites are an amino acid, a vitamin, an antibiotic, a carotenoid.

The method of the invention is suitable for the mutagenesis of any polynucleotide of interest.

In one embodiment of the invention, the polynucleotide of interest comprises a gene encoding a polypeptide. Said polypeptide may for instance be a structural protein, a peptide hormone, a growth factor, an antibody or an enzyme. The polypeptide may be produced intracellularly or may be secreted from the cell into the environment, for instance the culture medium. The polynucleotide may comprise a single gene or may comprise a cluster of genes. Said cluster of genes may comprise genes encoding enzymes involved in the biosynthesis of a particular metabolite and/or genes encoding regulatory factors involved in the regulation of expression of one or more genes involved in production of a particular metabolite.

In another embodiment of the invention, the polynucleotide of interest may be a non-coding polynucleotide, for instance a regulatory region involved in the control of gene expression, on transcriptional and/or translational level. The process of the invention may also be applied to a polynucleotide comprising a gene (cluster) and corresponding regulatory regions.

The present invention further envisages production of a variant polypeptide by expressing a variant polynucleotide produced and selected according to the invention in a suitable host organism and, optionally, recovery of the produced polypeptide.

To this end, the selected polynucleotide is cloned in an expression vector of choice and transformed to a host organism of choice. Transformed host cells are selected from the untransformed background by any suitable means. The transformed cells are grown in a suitable culture medium and may further be screened for expression of the variant polynucleotide. Techniques for the transformation of host cells and for the selection of transformed cells are commonly known to the skilled person.

For production of the variant polypeptide on a larger scale, a transformed cell producing a suitable amount of the variant polypeptide of interest may be cultured under conditions conducive to the production of said polypeptide. Optionally, the polypeptide may be recovered from the culture medium and/or form the host organism. Depending on its further use, recovery of the variant polypeptide may include its formulation in a suitable liquid or solid formulation, and/or its immobilization.

### BRIEF DESCRIPTION OF THE DRAWINGS

FIG. 1. Schematic illustration of the BERE recombination technique.

FIG. 2. Blunt-end restriction enzyme fragmentation used for the BERE recombination method.

FIG. 3. Agarose gel electrophoresis of the reassembly reaction. The arrow indicates DNA bands of the appropriate size (˜1 kb).
1: Marker
2: Reassembly (+Ampligase), 15 cycles
3: Reassembly (+Ampligase), 30 cycles
4: Starting material (all restriction fragments

FIG. 4. Typical results of the conversion activities of a group of mutants from the KKN05 library selected after the first MTP analysis.

### Experimental

MTP Screening

Single colonies of the library to be screened were inoculated in individual wells of microtiter plates (MTP's) filled with SE liquid medium (containing bacto tryptone 10 g/l, bacto yeast 5 g/l and NaCl 5 g/l), supplemented with ampicillin at a final concentration of 100 μg/ml. If required, arabinose inducer was added (final concentration 0.002%). Normal growth conditions were at 37° C.; induced growth conditions were at 28° C. and 280 rpm. 50 μl of the 20-24 hour grown cultures were incubated with D,L-α-methylphenylglycine amide (Femam) at 55° C. in deepwell plates. After 2.5 hours off incubation the amidase activity of the culture broth was measured by measuring the amount of formed L-α-methylglycine (Femac).

CFE Screening

Cell-free extracts (CFE's) were prepared using a bacterial protein extraction reagent according to the manufacturer's instructions (BPER, Pierce, Rockford, Ill. USA) and their L-amidase activity was measured.

Amidase Activity Assay

Amidase activity was measured as conversion activity from Femam to Femac. Detection occurred by NMR.

### EXAMPLE 1

Preparation and Screening of an Error-Prone Library of O. anthropi L-Amidase

An error-prone PCR was performed on the Ochrobactrum anthropi L-amidase gene (see SEQ ID No 1) using the Diversify™ PCR Random Mutagenesis kit from Clontech (Palo Alto, Calif. USA) according to the manufacturer's instructions. The PCR products were cloned in the EagI/HindIII sites of the vector pBAD/Myc-H is C (Invitrogen Corporation, Carlsbad, Calif. USA) and transformed to E Coli Top10F cells (Invitrogen Corporation, Carlsbad, Calif. USA). Clones were first screened on MTP and CFE's of a subset of clones were further screened (see Experimental). Improved mutants were sequenced to determine the modified position(s). The modified positions of seven improved mutants are indicated hereinafter: V52A, F93V, T143A, T193P, N212D, N981/L124P, K138R/G234V (see SEQ ID No 2).

### EXAMPLE 2

Recombination of improved mutants by BERE recombination

An oultline of the blunt-ended restriction enzyme (BERE) method is given in FIG. 1.

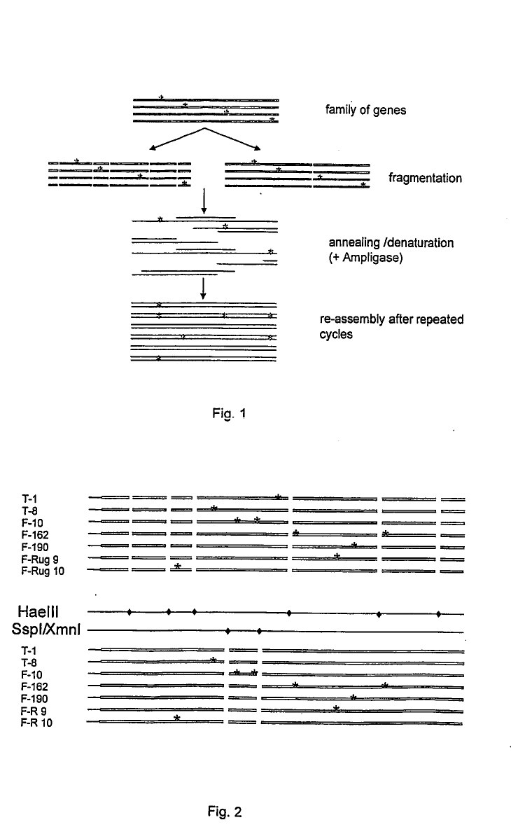

DNA of the seven mutant L-amidase genes as described in Example 1 was either digested with XmnI/SspI or with HaeIII. Two out of the total nine mutations were still located on one fragment after restriction enzyme fragmentation and therefore could not be recombined separately (see FIG. 2).

The fragments of both digestions were mixed and used for a reassembly reaction using Ampligase (Epicentre Technologies, Madison, Wis. USA). As can been seen in FIG. 3, already after 15 cycles of denaturation and annealing a DNA of the appropriate size appears.

The DNA product of the Ampligase-induced reassembly reaction was subsequently used as template for an error-prone PCR (EP-PCR).

For this experiment mild EP conditions designed to generate an average of around 1 basepair substitution per gene were used. The DNA products were cloned in the EagI/ HindIII sites of the pBAD/Myc-H is C vector and transformed to E. coli Top10F cells.

Of the resulting KKN05 library 5000 mutants were screened in MTP as described in Experimental.

DNA of the L-amidase genes of 10 randomly picked mutants from the KKN05 library was sequenced. The results of the sequence analysis are presented in Table 1. From the sequence results of the randomly picked mutants a recombination frequency of the mutations of at least 20% and an error prone frequency of 0.6 mutations/gene was calculated.

A large group of mutants selected from the MTP screening was tested in a secondary screening. In this screening, CVE's and different dilutions thereof were analysed for conversion activity by NMR. The conversion/μl was calculated and corrected for the amount of protein. Subsequently the conversion/μl/mg protein of the mutants was compared with conversion/μl/mg protein of the wild type, and the activity improvement was determined.

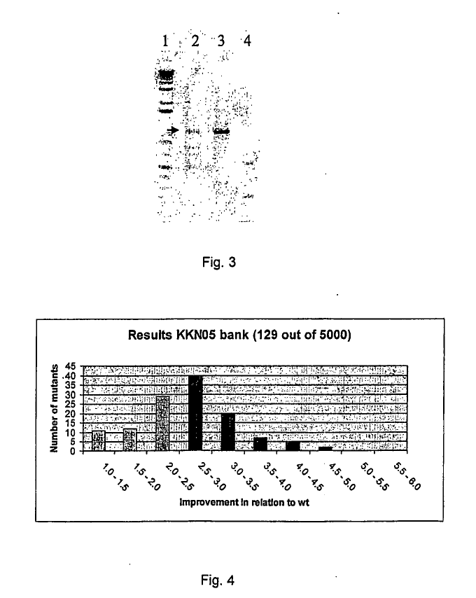

In FIG. 4 the overall results of selected mutants from the KKN05 library are presented.

After one round of BERE recombination, the specific activity of the mutants was substantially increased (FIG. 4). The mutant with the highest improvement turned out to be 4-5 times more active than wild type.

Subsequently DNA of the L-amidase genes of the top 10-improved mutants from the KKN05 library was sequenced. The results of the sequence analyses are presented in Table 1.

Five mutants turned out to be unique. Within two mutants additional mutations caused by EP PCR was demonstrated.

TABLE 1
Sequence result of 10 randomly picked and 10 selected colonies from the
KKN05 library. At the top the 7 EP mutants that were used as starting material for BERE
recombination. The conversion activity is indicated relative to wild type.
Positions 410, 500 and 842 relate to a point mutation without a change in amino acid.

| | | | | | | | | | | EP | Conversion |
|---|---|---|---|---|---|---|---|---|---|---|---|
| | Mutations | | | | | | | | | Mutations | activity |
| WT | | | | | | | | | | | 1 |
| T1 | | | | | | T143A | | | | | 1.8-2.3 |
| T8 | | F93V | | | | | | | | | 1.5 |
| F10 | | | N98I | L124P | | | | | | | 1.4 |
| P162 | | | | | K138R | | | | G234V | | 1.3 |
| P190 | | | | | | | | N212D | | | 1.1 |
| R9 | | | | | | | T193P | | | | 1.4 |
| R10 | V52A | | | | | | | | | | 2.0-2.5 |
| KKN05-1 | | | | | | | T193P | | | | |
| KKN05-2 | | | | | | T143A | | N212D | | | |
| KKN05-3 | | | | | | | | | G234V | N265S | |
| KKN05-4 | | | | | | | | N212D | | | |
| KKN05-5 | | | | | | | | | | S155P | |
| KKN05-6 | | | | | | | | | G234V | S50G | |
| KKN05-7 | | | | | | | | N212D | | P1593 | |
| KKN05-8 | | | | | | | | | G234V | I83V | |
| KKN05-9 | V52A | | | | | | | | | | |
| KKN06-10 | | F93V | | | | | T193P | | | | |
| F404 | | F93V | | | | | T193P | | | | 4.7 |
| F514 | | F93V | | | | | T193P | | | | 4.4 |
| F444 | V52A | | | | | T143A | | | | G42S, 410 | 4.3 |
| F632 | | | N98I | L124P | | | T193P | | | | 4.1 |
| F471 | V52A | | | | | T143A | | | | G42S, 410 | 4 |
| F620 | | | | | | | T193P | | | | 3.9 |
| F496 | | F93V | | | | | T193P | | | | 3.7 |
| F470 | | F93V | | | | | T193P | | | 500, 842 | 3.7 |
| F523 | V52A | | N98I | L124P | | | T193P | | | | 3.6 |
| F466 | | F93V | | | | | T193P | | | | 3.6 |

1. A process for the preparation of a variant polynucleotide having a desired property, comprising:
subjecting a population of polynucleotides to separate digestions with a restriction enzyme;
combining the digests;
applying one or more cycles of denaturation, annealing and reassembly in the presence of a ligase;
optionally amplifying the reassembled polynucleotides;
preparing a library of the resulting variant polynucleotides;
screening said library of variant polynucleotides for a variant polynucleotide with a desired property.

2. The process of claim 1, wherein the population of polynucleotides displays homology of at least 70%.

3. The process of claim 1, wherein the population of polynucleotides is selected from the group consisting of a population of different mutants of a parental polynucleotide and a population of different members of a gene family.

4. The process of claim 1, wherein the ligase ligates single-strand nicks in a double stranded polynucleotide.

5. The process of claim 1, wherein the ligase substantially does not ligate blunt-ended polynucleotide fragments.

6. The process of claim 1, wherein the ligase is a thermostable ligase.

7. The process of claim 1, wherein the amplification of the reassembled polynucleotides is performed under error-prone conditions.

8. The process of claim 1, wherein the polynucleotide comprises one or more gene(s) encoding a polypeptide.

9. The process of claim 9, wherein the polypeptide is involved in the biosynthetic pathway of a primary or secondary metabolite.

10. A process for the production of a variant polypeptide comprising expressing the variant polynucleotide prepared according to the process of claim 1 in a suitable host and, optionally, recovering the produced polypeptide.

11. A process for the production of a primary or secondary metabolite comprising expressing the variant polynucleotide prepared according to the process of claim 9 in a suitable host and, optionally, recovering the produced metabolite.

12. The process of claim 1 wherein said enzyme generates blunt-ended fragments.

13. The process of claim 2 wherein said homology is at least 75%.

14. The process of claim 13 wherein said homology is at least 80%.

15. The process of claim 14 wherein said homology is at least 85%.

16. The process of claim 15 wherein said homology is at least 90%.

17. The process of claim 16 wherein said homology is at least 95%
